(19) United States
(12) Patent Application Publication
(10) Pub. No.: US 2024/0300867 A1
(43) Pub. Date: Sep. 12, 2024

(54) BREVUNDIMONAS AND USE THEREOF IN TEA PLANT PLANTING

(71) Applicant: ANHUI AGRICULTURAL UNIVERSITY, Hefei (CN)

(72) Inventors: Xianchen Zhang, Hefei (CN); Ziwen Zhou, Hefei (CN); Wenya Liang, Hefei (CN); Yeyun Li, Hefei (CN); Zhougao Shen, Hefei (CN); Jiayue Jiang, Hefei (CN)

(21) Appl. No.: 18/407,310

(22) Filed: Jan. 8, 2024

(30) Foreign Application Priority Data
Mar. 7, 2023 (CN) 202310232873.4

(51) Int. Cl. C05F 11/08; A01G 29/00; C05G 5/23; C12N 1/20; C12R 1/01

(52) U.S. Cl. CPC C05F 11/08; A01G 29/00; C05G 5/23; C12N 1/205; C12R 2001/01

(57) ABSTRACT

The application includes a Brevundimonas and a use thereof in tea plant planting, belonging to the technical field of microorganisms. The Brevundimonas is Brevundimonas sp. AnNong-1 which is preserved in China Center for Type Culture Collection with a preservation number CCTCC M 20221537. The culture solution of the strain can significantly increase the function of the total amount of L-Theanine in first and second leaves of tea plants after being inoculated around a distance of about 5 cm from the roots of tea seedlings. The Brevundimonas sp. AnNong-1 of the present invention is easy to operate in fermentation conditions and can also be used for preparing a natural microbial fertilizer, which can significantly increase the content of L-Theanine without greatly affecting the content of tea polyphenol, thereby providing a choice for the tea market. The microbial fertilizer is an environmental-friendly fertilizer, and can greatly reduce environmental pollution.

### CROSS REFERENCE TO RELATED APPLICATIONS

The present invention claims priority to Chinese Patent Application No. 202310232873.4 filed with the China Patent Office on Mar. 7, 2023, entitled “Brevundimonas and use thereof in tea plant planting”, the disclosure of which is incorporated by reference herein in its entirety.

### INCORPORATION BY REFERENCE

sequence listing provided in the file entitled 1218-230476US-SQL.xml, which is an Extensible Markup Language (XML) file that was created on Apr. 30, 2024, and which comprises 4,940 bytes, is hereby incorporated by reference in its entirety.

### TECHNICAL FIELD

The present invention belongs to the technical field of microorganisms, particularly relates to a Brevundimonas and a use thereof in tea plant planting.

### BACKGROUND ART

Brevundimonas is a type of bacteria that are widely present in water, soil, plants and human body. Brevundimonas belongs to Brevundimonas family and Brevundimonas genus and is a type of Gram-positive bacteria that can produce resistant endospores. The cell is rod-shaped and covered with a large amount of DAP-Ca on the outer layer. Its cortex is located between a core and a spore shell and is rich in peptidoglycans. The core is a highly concentrated and inert chromosome. The outer wall of the outermost layer is a layer of peptidoglycan wall, and the components of one or more layers are spore coats of proteins. Since the spore has a thick multi-layer structure having a low water content, Brevundimonas has strong refractivity, is not easily colored by dyes, has good stability, oxidation resistance, extrusion resistance and high temperature resistance, can withstand a high temperature of 60° C. for a long time, can survive for 20 min at the temperature of 120° C., and is resistant to acid and alkali so as to maintain activity in a gastric acidic environment, which may be related to a large amount of pyridine dicarboxylic acid which is unique in spores. Meanwhile, Brevundimonas also has broad-spectrum bacillus activity, which can produce bacteriocins to inhibit pathogenic bacteria.

According to the present invention, Brevundimonas sp. AnNong-1 was isolated and obtained from the rhizosphere soil of a tea garden in Likou Town, Qimen County, Huangshan City, Anhui Province. The bacterial colonies isolated from the strains were cultured for 3-4 days at 28° C. in a potassium solubilizing culture medium, which has a diameter of 8-13 mm, then the bacterial colonies were cultured by activation or purification for 24 hours in an LB culture medium to obtain a diameter of 8-10 mm, and the diameter of the bacterial colonies after culture of 3 days reached 15-20 mm.

L-Theanine is an amino acid unique in tea leaves, with a chemical formula of C₇H₁₄N₂O₃, accounting for 1%-2% of the weight of dry tea leaves. L-Theanine was firstly isolated from green tea in 1950. It is a characteristic amino acid in tea leaves and is also one of flavor substances in tea leaves, which is strongly positively correlated with the quality of green tea, with a correlation coefficient reaching 0.787-0.876. Among more than 20 amino acids present in tea leaves, L-Theanine accounts for approximately 50%-60% of the total content of amino acids in tea leaves. It has been found through research that L-Theanine has not been found in other plants except that trace amounts of L-Theanine have been detected in plants such as camellia sasanqua, mushrooms and sasanqua.

L-Theanine is an important quality parameter in tea leaves, and the content of L-Theanine in green tea is an important component making tea refresh. There are no reports on how strains can increase the content of L-Theanine in tea leaves currently. In the present patent, strains that can increase the total content of L-Theanine were screened, which lays the foundation for the development of microbial fertilizers.

### SUMMARY OF THE INVENTION

The objective of the present invention is to provide a Brevundimonas and a use thereof in tea plant planting. After root irrigation treatment is performed on a tea plant with the strains, the total content of L-Theanine in tea leaves can be increased without increasing the content of tea polyphenols, and therefore Brevundimonas can be used for preparing a microbial fertilizer. The Brevundimonas had been preserved in China Center for Type Culture Collection (Wuhan University, Wuhan City, Hubei Province, Postal Code: 430072, telephone number: 027-68754052) with a preservation number CCTCC M 20221537 on Oct. 11, 2022, and its Latin name is Brevundimonas sp. AnNong-1.

The Brevundimonas sp. AnNong-1 bacterial colony preserved by the present invention is characterized in that it is white and round and has a smooth and opaque surface and a complete edge.

The gene sequence of the Brevundimonas sp. AnNong-1 is shown in SEQ ID NO: 1.

Meanwhile, the present invention provides a use of the newly screened Brevundimonas sp. AnNong-1, which is specifically as follows:

1. The Brevundimonas sp. AnNong-1 is used for root irrigation treatment of tea plants, which can increase the total content of L-Theanine in tea leaves.

2. The Brevundimonas sp. AnNong-1 is used for preparing a microbial fertilizer which increases the content of L-Theanine in tea leaves.

Meanwhile, the present invention also provides a method for increasing the total content of L-Theanine in tea leaves based on Brevundimonas sp. AnNong-1, comprising:
(1) inoculating activated Brevundimonas sp. AnNong-1 to an LB liquid culture medium to undergo shaking culture in a constant-temperature shaker at 28-30° C. to obtain a bacterial solution of Brevundimonas sp. AnNong-1;
(2) Pouring the bacterial solution of Brevundimonas sp. AnNong-1 on the roots of tea plants;
(3) when the tea plants grow to maturity, a significant increase in the tea content of its tea leaves is obtained; wherein, the Brevundimonas sp. AnNong-1 is preserved with a preservation number CCTCC M 20221537.

Meanwhile, the present invention also provides a microbial fertilizer promoting the synthesis of L-Theanine based on Brevundimonas sp. AnNong-1. Specifically, the microbial fertilizer comprises a bacterial solution of Brevundimonas sp. AnNong-1, and the Brevundimonas sp. AnNong-1 is preserved with a preservation number CCTCC M 20221537.

Wherein, the total number of bacterial colonies of the bacterial solution of Brevundimonas sp. AnNong-1 is 0.5-3.0×10⁸ colonies/mL.

### Beneficial Effects

First, the Brevundimonas sp. AnNong-1 preserved by the present invention was isolated from a tea garden that produced vigorous and high-yield L-Theanine. After root irrigation treatment is performed on tea seedlings using Brevundimonas sp. AnNong-1 of the present invention, the total content of L-Theanine in tea leaves can be significantly increased. According to data measured by experiments, the total content of L-Theanine in young tea leaves is increased to about 1.49% and 1.96% (measured under the same standard curve) from less than 1.0% in control group after treatment with Brevundimonas sp. AnNong-1. It can be seen from different treatment calculation values that the Brevundimonas sp. AnNong-1 of the present invention increases the content of L-Theanine by nearly twice that of the control group. The inventors have not yet inquired any relevant reports on Brevundimonas sp. AnNong-1 increasing the total content of L-Theanine in tea leaves.

Second, the Brevundimonas sp. AnNong-1 of the present invention can adapt well to natural environment, and continuously not increase the content of tea polyphenol in tea leaves while increasing the total content of L-Theanine in tea leaves, so as to make an important contribution to the improvement of green tea quality. However, it is found by study on the secretions of Brevundimonas sp. AnNong-1 that the secretions of Brevundimonas sp. AnNong-1 contain amino acids and various organic acids. The detailed mechanism of increasing L-Theanine needs to be further researched.

Finally, the Bacillus AnNong-1 preserved by the present invention can be used as an organic biological fertilizer, which provides new ideas for tree cultivation and can help to plant high-quality green tea with rich flavor. Meanwhile, the strains of the present invention are simple in culture condition, low in culture cost, and have a broad market prospect.

### BRIEF DESCRIPTION OF THE DRAWINGS

FIG. 1 is a bacterial colony chart of a Brevundimonas sp. AnNong-1 of the present invention.

FIG. 2 is a diagram showing physicochemical properties of Brevundimonas sp. AnNong-1 strains of the present invention.

FIG. 3 is a bar graph of a total content of L-Theanine in tea leaves.

FIG. 4 is a bar graph of a total content of tea polyphenol in tea leaves.

### DETAILED DESCRIPTION OF THE INVENTION

Next, the present invention will be described in detail in combination with examples. Unless otherwise specified, methods and technologies are all conventional methods and technologies.

### Example 1

The strains of Brevundimonas sp. AnNong-1 had been preserved in China Center for Type Culture Collection on Oct. 11, 2022, with a preservation number CCTCC M 20221537.

Where, other reagents used in examples of the present invention were all analytically pure.

The Brevundimonas sp.AnNong-1 preserved by the present invention was isolated from the rhizosphere soil of a tea garden in Likou Town, Qimen County, Huangshan City, Anhui Province. Specific steps were as follows:

1) Screening of strains: 10.0 g of a fresh soil sample was weighed and placed in a 250 mL conical flask containing 90 mL of sterile water, then shaken for 30 min in a full-temperature shaking incubator at 28-30° C. under 180 r min-1, so a soil suspension was prepared after sufficiently and evenly mixing. 100 μL of the soil suspension was added into a 1.5 mL sterilized centrifuge tube containing 900 μL of sterile water and then diluted to 10-2, 10-3 and 10-4 in sequence using a ten-fold gradient dilution method. 50 μL of diluted solutions were respectively coated onto a solid culture medium comprising the following components: 10.0 g of glucose, 0.2 g of MgSO₄·7H₂O, 0.2 g of Na₂HPO₄, 5.0 g of CaCO3, 0.2 g of NaCl, 0.2 g of CaSO₄·2H₂O, 2.5 g of potassium feldspar powder (K₂O·Al₂O₃·6SiO₂), 20 g of agar powder, 1000 mL of H₂O, and with a pH of 7.2-7.5.

2) Culture of strains: the culture medium was cultured for 72-96 h in an incubator at 28-30° C. Single bacterial colonies were picked and placed in an LB solid culture medium comprising the following components: 10 g of peptone, 5 g of yeast powder, 5 g of sodium chloride, 1000 mL of water and 20 g of agar powder, and with a pH of 7.0-7.2. After strains were purified, a bacterial colony where a bacterial circle had a maximum diameter was selected and preserved in −80° C. by using a 40% glycerol preservation method for future use.

3) Activation: target bacterial colony preserved in a glycerol tube was removed therefrom and activated in an LB solid culture medium, cultured for 2-3 d at 28-30° C.

4) Morphological characteristics and physiological and biochemical characteristics of strains

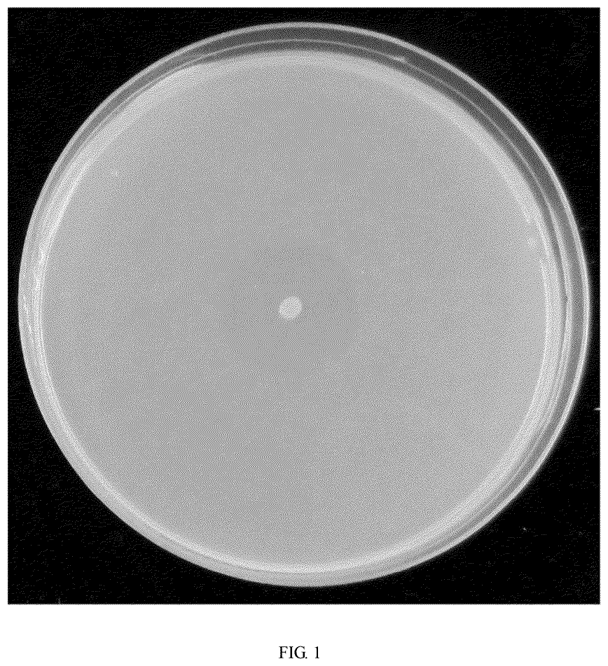

Morphological characteristics: the bacterial colony was white and round and had a smooth and opaque surface and a complete edge. Its morphology on the LB culture medium is shown in FIG. 1.

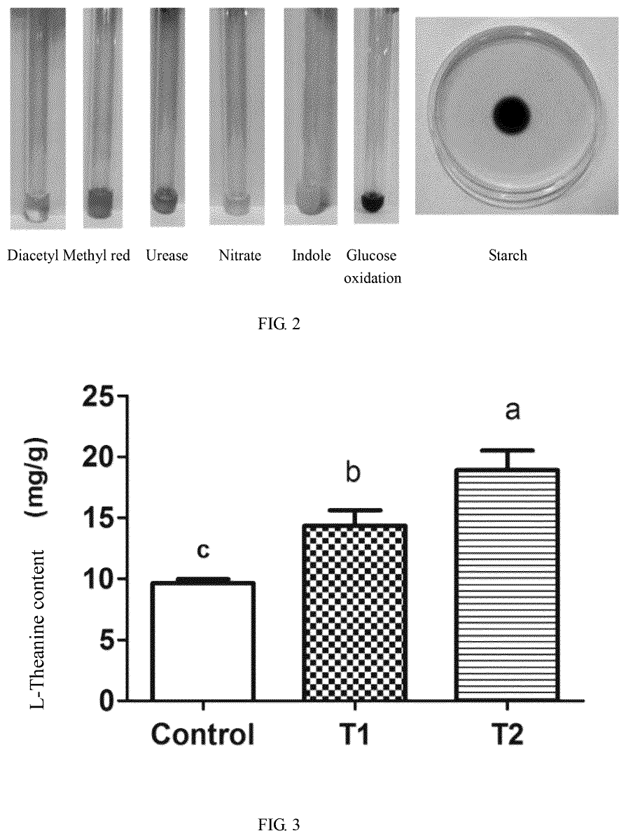

Physiological and biochemical characteristics: by physiological and biochemical determination, the target strains were positive for methyl red test; negative for indole test; negative for diacetyl test; negative for urease test; negative for nitrate reduction test; positive for glycolysis test; positive for starch hydrolysis test. The results are shown in FIG. 2.

TABLE 1
Physiological and biochemical features of strains

| Test | Positive/negative |
|---|---|
| Methyl red test | + |
| Indole test | − |
| Diacetyl test | − |
| Urease test | − |
| Nitrate reduction test | − |
| Glycolysis test | + |
| Starch hydrolysis test | + |

Note:
“+” positive;
“−” negative

5) Extraction of strain DNA: the strains were inoculated in an LB liquid culture medium and subjected to shaking culture for 24-36 h at 28° C. under 180 r·min⁻¹; 1 mL of the bacterial solution was put into a 1.5 mL Eppendorf centrifuge tube for centrifugation for 3 min at 8000 rpm, after which the thalli were collected, and strain DNA was extracted according to a kit method (Shanghai Shenggong Biotechnology Co., Ltd).

6) Identification of strains

PCR amplification of 16S rDNA genes: forward primer 27F: (SEQ ID NO: 2); reverse primer 1492R: (SEQ ID NO: 3).

The PCR reaction system is shown in Table 2:

TABLE 2
PCR reaction system

| Component | Reaction solution Volume V (μL) |
|---|---|
| Template DNA | 1 |
| Primer 1 | 1 |
| Primer 2 | 1 |
| Master Mix | 12.5 |
| ddH₂O | 9.5 |

The reaction procedure is shown in Table 3.

TABLE 3
Reaction procedure table

| Reaction procedure | Temperature (° C.) | Time (min) | Cycle times |
|---|---|---|---|
| Initial denaturation | 94 | 4 | 1 |
| Denaturation | 94 | 0.5 | 30 |
| Renaturation | 55 | 0.5 | 30 |
| Extension | 72 | 1.5 | 30 |
| Terminal extension | 72 | 10 | 1 |
| Preservation | 4 | ∞ | 1 |

The bacterial 16S rDNA sequence was subjected to PCR amplification, and the amplified product was sequenced and then compared with a known 16S rDNA sequence in EzBioCloud. It was identified as Brevundimonas genus.

### Example 2

Brevundimonas sp.AnNong-1 preserved in Example 1 was cultured for 48 h in an LB culture medium, during which the secretions of Brevundimonas sp.AnNong-1 were taken at regular intervals. The contents of amino acids and organic acids in the secretions were detected. The results are shown in Table 4 and Table 5.

TABLE 4
Amino acids of Brevundimonas sp. AnNong-1 preserved by the present
invention

| Amino acid | Brevundimonas sp. AnNong-1 fermentation time/h | | | | | | | |
|---|---|---|---|---|---|---|---|---|
| (ng/μL) | 2 h | 4 h | 8 h | 16 h | 24 h | 28 h | 32 h | 48 h |
| Asp (aspartic acid) | 0.29 | 0.29 | 0.46 | 0.05 | — | — | — | — |
| Thr (threonine) | 1.08 | 1.07 | 1.37 | 0.43 | — | — | — | — |
| Ser (serine) | 1.33 | 1.30 | 1.67 | 0.14 | — | — | — | — |
| Glu (glutamic acid) | 4.96 | 4.82 | 4.2 | 0.99 | 0.05 | — | — | — |
| Gly (glycine) | 10.38 | 10.03 | 8.93 | 4.88 | 0.43 | — | — | — |
| Ala (alanine) | 2.92 | 2.74 | 2.71 | 0.23 | — | — | — | — |
| Cit (citrulline) | 0.98 | 0.94 | 0.35 | — | — | — | — | — |
| Val (valine) | 2.27 | 2.19 | 2.27 | 0.5 | — | — | — | — |
| Met (methionine) | 0.66 | 0.65 | 0.58 | — | — | — | — | — |
| Ile (isoleucine) | 0.95 | 0.92 | 1.1 | — | — | — | — | — |
| Leu (leucine) | 1.57 | 1.44 | 1.56 | — | — | — | — | — |
| Tyr (tyrosine) | 0.75 | 0.74 | 0.73 | — | — | — | — | — |
| Phe (phenylalanine) | 2.05 | 1.89 | 1.5 | 0.27 | 0.14 | — | — | — |
| Orn (ornithine) | 0.11 | 0.10 | 0.12 | — | — | — | — | — |
| Arg (argnine) | 2.11 | 1.70 | 2.49 | — | — | — | — | — |
| Pro (proline) | 0.74 | 0.64 | 0.6 | — | — | — | — | — |

TABLE 5
Organic acid secretion table of Brevundimonas

| Organic | | | | | | | | |
|---|---|---|---|---|---|---|---|---|
| acid | time/h | | | | | | | |
| (μg/mL) | 2 h | 4 h | 8 h | 16 h | 24 h | 28 h | 32 h | 48 h |
| Oxalic | — | — | — | — | — | — | — | — |
| acid | | | | | | | | |
| Tartaric | — | — | — | 195.82 | 206.50 | 366.79 | 435.29 | 441.80 |
| acid | | | | | | | | |
| Pyruvic | — | — | 120.29 | — | — | — | — | — |
| acid | | | | | | | | |
| Citric | — | 14.61 | — | 14.61 | — | 51.21 | 52.92 | 53.01 |
| acid | | | | | | | | |
| Malic | — | — | 33.39 | 68.31 | 155.19 | 71.36 | — | — |
| acid | | | | | | | | |
| Glacial | 15.94 | 23.66 | 16.06 | 12.63 | 11.35 | 12.05 | 9.33745 | 4.13 |
| acetic | | | | | | | | |
| acid | | | | | | | | |
| Succinic | 46.26 | 48.18 | 44.46 | 50.51 | 53.13 | 51.21 | 52.92 | 53.01 |
| acid | | | | | | | | |
| Gluconic | — | — | — | — | — | — | — | — |
| acid | | | | | | | | |

It can be seen from the data of Table 4 and Tale 5 that bacillus AnNong-1 preserved in Example 1 secreted amino acids and organic acids during the culture.

### Example 3

This example is an example in which Brevundimonas sp. AnNong-1 preserved by the present invention is used for increasing the content of L-Theanine in tea leaves.

First, potting treatment of tea seedlings specifically comprises the following steps:

1) Activation: Brevundimonas sp. AnNong-1 deposited in glycerol was taken, the glycerol tube was shaken up and down so that the bacterial solution and glycerol were evenly distributed. Then a fired and cooled inoculating loop was used to dip the bacterial solution in the glycerol tube. The inoculating loop dipped with the bacterial solution was streaked and activated on an LB solid culture medium, then the LB solid plate with activated strains was sealed with a sealing film and marked for strain name and date using a marker. The formulation of the LB solid culture medium was as follows: 10 g of peptone, 5 g of yeast powder, 5 g of sodium chloride, 1000 mL of water and 20 g of agar powder, and the culture medium had a pH of 7.0-7.2.

2) Culture: the streaked and activated strain sealing plate was placed inversely in a constant temperature incubator at 28-30° C. to be cultured for 2-3 d.

3) Preparation of bacterial solution: after single bacterial colonies were grown on the activated plate, a single bacterial colony was picked using an inoculating loop to be inoculated in a 250 ml conical flask containing 100 mL of an LB liquid culture medium (10 g of peptone, 5 g of yeast powder, 5 g of sodium chloride and 1000 mL of water, and pH 7.0-7.2) and subjected to shaking culture for about 10-12 h in a constant-temperature shaker at 28-30° C. under 180 r·min-1. The OD₆₀₀ value of the strain culture solution was determined using a preheated ultraviolet spectrophotometer, and an LB liquid culture medium was used as a control. When the OD₆₀₀ value of the strain culture solution was 0.45-0.50 (total number of bacterial colonies was 2.3-2.8×10⁸), strain amplification culture on the constant-temperature shaker was stopped, and the strain culture solution at this moment was a liquid inoculum of strains. The cultured strain solution was transferred to a 550 mL plastic bottle and marked for future use.

4) Bacterial application treatment: tea seedlings were started to be subjected to bacterial application treatment on Dec. 1, 2020. Namely, weeds and fallen leaves around the tea seedlings were cleared, and then a soil shovel was used to dig pits 3-5 cm around the tea seedlings at a depth of 10-13 cm from the ground. The dug soil was piled up according to the contour of the pit, then 50 mL of the bacterial solution which was put in a 550 mL plastic bottle after being cultured was applied every 3.5 kg of soil per pot. Root irrigation treatment was performed around the tea seedlings using a mode of applying the same volume of water as a control. Finally, soil around the pits was covered again on the surface of the bacterial solution after bacterial application, so that the bacterial solution was completely covered, thereby avoiding direct sunlight in summer.

5) In May 1, 2021, the first and second leaves of the treated tea seedlings were sealed in marked self-sealing bags and put in a dry ice box, and then stored in a refrigerator at −80° C. for the determination of total L-Theanine content.

6) Determination of total L-Theanine content comprises the following specific operation steps:

(1) After first and second fresh tea leaves were evenly mixed under the same treatment, the mixture was ground with liquid nitrogen, then about 0.1000 g of mixed fresh tea leaves were weighed and put in a 5 mL centrifuge tube, and the mass (g) of weighed and ground fresh tea leaves was recorded.

(2) 2 mL of distilled water was added into a 5 mL centrifuge tube containing fresh tea leaves, shaken and then placed in water bath at 100° C. for 30 min, during which the centrifuge tube was shaken up and down every 10 min.

(3) The above fresh tea leaves were centrifuged for 15 min at 15000 rpm and passed through a 0.22 μm water-system membrane twice and then transferred to an injection vial.

(4) Standard L-Theanine stock solution: 0.05 g of L-Theanine (accurate to 0.0001 g) was weighed and dissolved with distilled water and then transferred into a 50 mL volumetric flask and diluted to scale and evenly mixed. This solution contained 1 mg of L-Theanine per milliliter.

(5) Preparation of standard L-Theanine use solutions: 0.0, 0.1, 0.2, 0.5, 1, 1.5, 2.0, 5.0 and 10.0 mL of standard L-Theanine stock solutions were accurately weighed respectively, diluted with distilled water to a constant volume of 10 mL to obtain standard L-Theanine use solutions with concentrations of 0.0, 0.01, 0.02, 0.05, 0.10, 0.15, 0.20, 0.50 and 1.0 mg/mL, respectively.

(6) L-Theanine was determined using the following elution procedure shown in Table 6 using flow phases (phase A: distilled water, phase B: pure acetonitrile) under the conditions of Agilent high-performance liquid chromatograph (1260), Waters E2695 HPLC (Waters, USA) and a Phenomenex C18 reverse chromatography column (5 μm, 250 mm×4.6 mm, Phenomenex, Los Angeles, USA):

TABLE 6
Elution procedure table for L-Theanine determination

| | | Flow phase | | Injection | Detection | Detection |
|---|---|---|---|---|---|---|
| Time | Flow rate | Phase | Phase | volume | temperature | wavelength |
| (min) | (mL/min) | A | B | (μL) | (° C.) | (nm) |
| 0 | 1 | 95% | 5% | 10 | 37 | 278 |
| 2 | | 80% | 20% | | | |
| 14 | | 75% | 25% | | | |
| 20 | | 58% | 42% | | | |
| 22 | | 42% | | | | |
| 28 | | 0% | 100% | | | |
| 31 | | 100% | | | | |
| 35 | | 95% | 5% | | | |
| 38 | | 95% | 5% | | | |

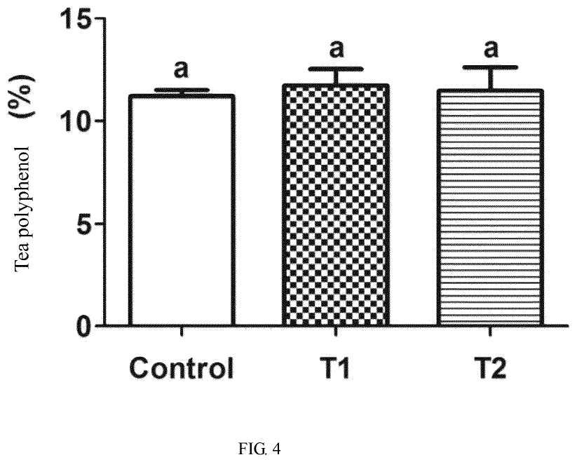

Tea plants were divided into three groups for treatment, which were respectively irrigated with an LB culture medium (control group), Brevundimonas sp. AnNong-1 with an OD600 value concentration of 0.45-0.50 (T1 group), Brevundimonas sp. AnNong-1 with an OD600 value concentration of 0.80-0.90 (T2 group). The contents of L-Theanine and tea polyphenol in three groups were respectively measured and shown in Tables 7 and 8 and FIGS. 3 and 4. It can be seen from the results of total L-Theanine content in FIG. 3 and Table 7 that after treatment, the total content of L-Theanine in young tea leaves is increased to about 1.44% and 1.89% from less than 1.0% in control group. Accordingly, Brevundimonas sp. AnNong-1 preserved by the present invention significantly increases the total content of L-Theanine in leaves.

TABLE 7
Total L-Theanine content table

| Total L-Theanine | | | |
|---|---|---|---|
| content | Control | T1 | T2 |
| Sample 1 | 9.62 | 11.51 | 15.73 |
| Sample 2 | 10.21 | 16.38 | 16.30 |
| Sample 3 | 9.08 | 11.37 | 21.65 |
| Sample 4 | | 11.95 | 23.78 |
| Sample 5 | | 16.55 | 17.02 |
| Sample 6 | | 18.34 | |

The content of tea polyphenol in tea leaves was measured through a standard curve by extracting tea polyphenol in fresh tea leaves with methanol. The specific method was as follows:

(1) The first and second leaves of fresh tea leaves under the same treatment were subjected to vacuum freeze drying treatment, then evenly mixed and ground with liquid nitrogen. 0.05 g of evenly ground tea sample from mixed fresh tea leaves were accurately weighed and put in a 5 mL centrifuge tube.

(2) 1.25 mL of a preheated 70% methanol solution was added into the 5 mL centrifuge tube containing tea samples, the centrifuge tube was covered and placed on a vortex apparatus for 1 min of vibration so that the tea samples were fully moisten and evenly mixed. And then the obtained mixture was immersed for 10 min in a 70° C. water bath (shake for 3 times up and down every 5 min) followed by cooling to room temperature.

(3) The immersed tea sample was centrifuged for 10 min at 3500 r/min, the supernatant after centrifuge was transferred to a 5 mL centrifuge tube. The partial rest tea residue was extracted once again according to step (2), extraction solutions were merged and diluted to a constant volume of 2.5 mL (the supernatant and dilution were both carried out using a quantification gun) for future use.

Preparation of test solution: 1.0 mL of a sample extraction solution after merging was transferred into a 100 mL volumetric flask and diluted to a scale with distilled water and evenly mixed for determination.

With reference to national standard (GB/T 8313-2008), a standard test solution and samples were determined. The phenolic hydroxyl group of tea polyphenol can be oxidized by folin-phenol, and its maximum absorption wavelength was displayed at 765 nm. Under this wavelength, the absorbances of the standard test solution and the sample solutions were measured. According to gallic acid working solution concentration in a range of 10-60 μg/mL, its corresponding absorbance value was measured to obtain a linear regression equation and a correlated coefficient (R2 is greater than 0.99). A sample measurement value was input into the linear regression equation to obtain the content of tea polyphenols in tea leaves. The measurement results are shown in FIG. 4 and Table 8. According to FIG. 4 and Table 8, it can be seen that the application of Brevundimonas sp. AnNong-1 preserved by the present invention has little effect on tea polyphenols.

TABLE 8
Total tea polyphenol content table

| Tea polyphenol | | | |
|---|---|---|---|
| content | Control | T1 | T2 |
| Sample 1 | 9.67 | 9.53 | 10.61 |
| Sample 2 | 11.41 | 11.99 | 16.00 |
| Sample 3 | 12.24 | 14.42 | 10.75 |
| Sample 4 | 11.75 | 13.16 | 10.02 |
| Sample 5 | 11.36 | 11.74 | 10.05 |
| Sample 6 | 10.92 | 9.57 | |

### Example 4

This example provides a biological organic fertilizer which is applied by a root irrigation method. The biological organic fertilizer comprises an AnNong-2 bacterial solution. The preparation method of the bacterial solution was as follows:

1) Activation: Brevundimonas sp. AnNong-1 deposited in glycerol was taken, the glycerol tube was shaken up and down so that the bacterial solution and glycerol were evenly distributed. Then a fired and cooled inoculating loop was used to dip the bacterial solution in the glycerol tube. The inoculating loop dipped with the bacterial solution was streaked and activated on an LB solid culture medium, then the LB solid plate with activated strains was sealed with a sealing film and marked for strain name and date using a marker. The formulation of the LB solid culture medium was as follows: 10 g of peptone, 5 g of yeast powder, 5 g of sodium chloride, 1000 mL of water and 20 g of agar powder, and the culture medium had a pH of 7.0-7.2.

2) Culture: the streaked and activated strain sealing plate was placed inversely in a constant-temperature incubator at 28-30° C. to be cultured for 2-3 d.

3) Preparation of bacterial solution: after single bacterial colonies were grown on the activated plate, a single bacterial colony was picked using an inoculating loop to be inoculated in a 250 mL conical flask containing a liquid culture medium (1 g/L of yeast powder, 5 g/L of sodium chloride, 10 g/L of starch, the temperature 35° C. and pH 6) and subjected to shaking culture for about 10-12 h in a constant-temperature shaker at 28-30° C. under 180 r·min-1. The content of strains in the culture solution was controlled as 0.5-3.0×10⁸ strains/mL, the bacterial solution at this moment was the biological organic fertilizer.

### Required Culture Mediums:

LB culture medium: 5 g/L of yeast powder, 10 g/L of tryptone, and 5 g/L of NaCl.
1.5 g of agar powder was added into every 100 mL of solid LB.
Fermentation culture medium: 1 g/L of bean pulp, 10 g/L of starch, 5 g/L of NaCl, pH=6.0
   Specific Steps were as Follows:

1. Activation of strains: strains were taken at −80° C., and properly shaken evenly before use. 200 μL of a bacterial solution was sucked and added into a 6 mL LB liquid culture medium, and then subjected to shaking culture for 10 h in a shaker at 28° C. under 200 rpm until the bacterial solution was turbid.

2. Streaking: the above activated bacterial solution was picked using an inoculating loop and streaked on a solid LB plate (four regions were streaked) and cultured for 13 h in an incubator at 28° C. until clear bacterial colonies were grown.

3. Preparation of mother solution: a single colony was picked from the above plate and put in a 100 mL LB liquid culture medium and then subjected to shaking culture for 13 h in a shaker at 28° C. under 200 rpm.

4. Inoculation: the mother solution was inoculated into a fermentation culture medium according to an inoculation amount of 4%, and then subjected to shaking culture for 10 h in the shaker at 35° C. under 200 rpm, so as to obtain a bacterial solution used for fertilizer application.

Potted tea seedlings before fertilizer application were dug, namely, a soil shovel was used to dig pits 3-5 cm around the tea seedlings at a depth of 10-13 cm from the ground. 50 mL of a bacterial solution after being cultured as a biological organic fertilizer was applied every 3.5 kg of soil per pot to perform root irrigation treatment around the tea plant, and then the root of the tea plant was covered with soil so as to complete bacterial application treatment.

When the biological organic fertilizer was used, the above biological organic fertilizer was irrigated at the root of the tea plant, which can promote the rapid growth of the tea plant and increase the content of L-Theanine in tea leaves.

### Example 5

Through gene sequencing, the gene sequence of the Brevundimonas sp. AnNong-1 preserved by the present invention was shown in SEQ ID NO: 1.

1. A Brevundimonas, wherein the Brevundimonas is Brevundimonas sp. AnNong-1 which is preserved in China Center for Type Culture Collection with a preservation number CCTCC M 20221537.

2. The Brevundimonas according to claim 1, wherein the gene sequence of the Brevundimonas is as follows: GCAGTCGACGAACTCTTCGGAGTTAGTGGCGGACGGGTGAGTA ACACGTGGGAACGTGCCTTTAGGTTCGGAATAACTCAGGGAAA CTTGTGCTAATACCGAATGTGCCCTTCGGGGGAAAGATTTATC GCCTTTAGAGCGGCCCGCGTCTGATTAGCTAGTTGGTGAGGTA AAGGCTCACCAAGGCGACGATCAGTAGCTGGTCTGAGAGGAT GATCAGCCACATTGGGACTGAGACACGGCCCAAACTCCTACGG GAGGCAGCAGTGGGGAATCTTGCGCAATGGGCGAAAGCCTGA CGCAGCCATGCCGCGTGAATGATGAAGGTCTTAGGATTGTAAA ATTCTTTCACCGGGGACGATAATGACGGTACCCGGAGAAGAA GCCCCGGCTAACTTCGTGCCAGCAGCCGCGGTAATACGAAGGG GGCTAGCGTTGCTCGGAATTACTGGGCGTAAAGGGAGCGTAG GCGGACATTTAAGTCAGGGGTGAAATCCCGGGGCTCAACCTCG GAATTGCCTTTGATACTGGGTGTCTTGAGTATGAGAGAGGTGT GTGGAACTCCGAGTGTAGAGGTGAAATTCGTAGATATTCGGAA GAACACCAGTGGCGAAGGCGACACACTGGCTCATTACTGACG CTGAGGCTCGAAAGCGTGGGGAGCAAACAGGATTAGATACCC TGGTAGTCCACGCCGTAAACGATGATTGCTAGTTGTCGGGATG CATGCATTTCGGTGACGCAGCTAACGCATTAAGCAATCCGCCT GGGGAGTACGGTCGCAAGATTAAAACTCAAAGGAATTGACGG GGGCCCGCACAAGCGGTGGAGCATGTGGTTTAATTCGAAGCA ACGCGCAGAACCTTACCACCTTTTGACATGCCTGGACCGCCAC GGAGACGTGGCTTTCCCTTCGGGGACTAGGACACAGGTGCTGC ATGGCTGTCGTCAGCTCGTGTCGTGAGATGTTGGGTTAAGTCC CGCAACGAGCGCAACCCTCGCCATTAGTTGCCATCATTTAGTT GGGAACTCTAATGGGACTGCCGGTGCTAAGCCGGAGGAAGGT GGGGATGACGTCAAGTCCTCATGGCCCTTACAGGGTGGGCTAC ACACGTGCTACAATGGCGACTACAGAGGGTTAATCCTTAAAAG TCGTCTCAGTTCGGATTGTCCTCTGCAACTCGAGGGCATGAAG TTGGAATCGCTAGTAATCGCGGATCAGCATGCCGCGGTGAATA CGTTCCCGGGCCTTGTACACACCGCCCGTCACACCATGGGAGT TGGTTCTACCCGAAGGCGCTGCGCTGACCGCAAGGGGGCAGC GACCACGTA.

3. A use of the Brevundimonas according to claim 1, wherein the Brevundimonas is used for root irrigation treatment of tea plants to increase the content of L-Theanine in tea leaves.

4. A use of the Brevundimonas according to claim 1, wherein the Brevundimonas is used for preparing a microbial fertilizer which increases the content of L-Theanine in tea leaves.

5. A method for increasing the content of L-Theanine in tea leaves, comprising:
(1) inoculating activated Brevundimonas sp. AnNong-1 to a liquid culture medium to undergo shaking culture for about 24 h at 28-30° C. under 180 rpm to obtain a bacterial solution of Brevundimonas sp. AnNong-1;
(2) performing root irrigation treatment around the root of tea plants with the bacterial solution of Brevundimonas sp. AnNong-1;
(3) the content of L-Theanine in tea leaves is significantly increased after spring tea plant grows new leaves;
wherein, the Brevundimonas sp. AnNong-1 is preserved with a preservation number CCTCC M 20221537.

6. The method for increasing the content of L-Theanine in tea leaves according to claim 5, wherein the liquid culture medium comprises yeast extract, NaCl and starch.

7. The method for increasing the content of L-Theanine in tea leaves according to claim 5, wherein the pH of the culture medium is 5.5-6.5.

8. A microbial fertilizer promoting the synthesis of L-Theanine, comprising a bacterial solution of Brevundimonas sp. AnNong-1 which is preserved with a preservation number CCTCC M 20221537.

9. The microbial fertilizer promoting the synthesis of L-Theanine according to claim 8, wherein the total number of bacterial colonies of the bacterial solution of Brevundimonas sp. AnNong-1 is 0.5-3.0×10⁸ colonies/mL.


SEQUENCE LISTING (3 sequences)

SEQ ID NO: 1 - DNA, 1332 bp, Brevundimonas sp.
  source 1..1332: mol_type = genomic DNA
gcagtcgacgaactcttcggagttagtggcggacgggtgagtaacacgtgggaacgtgcctttaggttcggaataactcagggaaacttgtgctaataccgaatgtgcccttcgggggaaagatttatcgcctttagagcggcccgcgtctgattagctagttggtgaggtaaaggctcaccaaggcgacgatcagtagctggtctgagaggatgatcagccacattgggactgagacacggcccaaactcctacgggaggcagcagtggggaatcttgcgcaatgggcgaaagcctgacgcagccatgccgcgtgaatgatgaaggtcttaggattgtaaaattctttcaccggggacgataatgacggtacccggagaagaagccccggctaacttcgtgccagcagccgcggtaatacgaagggggctagcgttgctcggaattactgggcgtaaagggagcgtaggcggacatttaagtcaggggtgaaatcccggggctcaacctcggaattgcctttgatactgggtgtcttgagtatgagagaggtgtgtggaactccgagtgtagaggtgaaattcgtagatattcggaagaacaccagtggcgaaggcgacacactggctcattactgacgctgaggctcgaaagcgtggggagcaaacaggattagataccctggtagtccacgccgtaaacgatgattgctagttgtcgggatgcatgcatttcggtgacgcagctaacgcattaagcaatccgcctggggagtacggtcgcaagattaaaactcaaaggaattgacgggggcccgcacaagcggtggagcatgtggtttaattcgaagcaacgcgcagaaccttaccaccttttgacatgcctggaccgccacggagacgtggctttcccttcggggactaggacacaggtgctgcatggctgtcgtcagctcgtgtcgtgagatgttgggttaagtcccgcaacgagcgcaaccctcgccattagttgccatcatttagttgggaactctaatgggactgccggtgctaagccggaggaaggtggggatgacgtcaagtcctcatggcccttacagggtgggctacacacgtgctacaatggcgactacagagggttaatccttaaaagtcgtctcagttcggattgtcctctgcaactcgagggcatgaagttggaatcgctagtaatcgcggatcagcatgccgcggtgaatacgttcccgggccttgtacacaccgcccgtcacaccatgggagttggttctacccgaaggcgctgcgctgaccgcaagggggcagcgaccacgta

SEQ ID NO: 2 - DNA, 20 bp, synthetic construct
  source 1..20: mol_type = other DNA
agagtttgatcctggctcag

SEQ ID NO: 3 - DNA, 19 bp, synthetic construct
  source 1..19: mol_type = other DNA
ggttaccttgttacgactt